(19) United States
(12) Patent Application Publication
(10) Pub. No.: US 2017/0181927 A1
(43) Pub. Date: Jun. 29, 2017

(54) MAGNETIC MOXIBUSTION T-SHIRT VEST

(71) Applicant: Zhongping Lou, Hangzhou (CN)

(72) Inventor: Zhongping Lou, Hangzhou (CN)

(21) Appl. No.: 15/275,378

(22) Filed: Sep. 24, 2016

(30) Foreign Application Priority Data
Dec. 28, 2015 (CN) 201521115674.2

(51) Int. Cl. A61H 39/00; A61N 2/06; A41D 27/02; A61N 2/00; A41B 1/08; A41D 1/04

(52) U.S. Cl. CPC A61H 39/00; A41B 1/08; A41D 1/04; A41D 27/02; A61N 2/008; A61N 2/06; A41B 2300/20; A41B 2300/328; A41B 2300/35; A41B 2400/324; A61H 2039/005

(57) ABSTRACT

The present invention is a magnetic moxibustion T-shirt vest which includes: a T-shirt vest noumenon and multiple magnetic stripes fixed and distributed on the noumenon. The described T-shirt vest noumenon includes posterior film, left anterior film, right anterior film and sleeves. The described multiple magnetic stripes are set on the described T-shirt vest noumenon posterior film, left anterior film, right anterior film and sleeves based on the preset primary spacing, and the described magnetic stripes include substrate and multiple magnets set on the substrate based on the preset secondary spacing. The described multiple magnets are fixed on the substrate based on unipolar direction or bipolar direction. The magnetic moxibustion T-shirt vest has achieved magnetotherapy on the whole upper body, and has the function of mitigating and treating shoulder and cervical spondylosis.

### REFERENCE TO RELATED APPLICATIONS

The present invention claims the priority date of the Chinese Patent Application No. 201521115674 which was filed on Dec. 28, 2015.

### FIELD OF THE INVENTION

The present invention generally relates to the field of magnetic therapy. More particularly, the invention relates to a magnetic moxibustion T-shirt vest.

### BACKGROUND OF THE INVENTION

With decrease of exercise time due to many factors including changes in lifestyle and workstyle, the morbidity rates due to cervical and shoulder spondylosis has increased and patients age faster, particularly those who work in the profession of officers, management staff, and customer service truck and vehicle drivers.

At present, conventional treatment for shoulder and cervical spondylosis is normally by mechanical traction, manual massage, sticking plaster, and other approaches. However, mechanical traction and manual massage can only be conducted In particular occasions, and only consecutive treatment can have certain efficacy. In this case, this approach brings lots of inconvenience to the work and life of commuters. Although sticking plaster can overcome the disadvantages of mechanical traction and manual massage, redness and allergy may be induced due to long-time sticking of plaster to the skin. Skin ulceration may also occur for severe cases. Moreover, sticking plaster can only relieve pain temporarily. Therefore, what is needed is a moxibustion vest in a shape of a t-shirt that drivers, for example, can wear during commute time to provide therapy for shoulder and cervical spondylosis. This vest can then provide consecutive therapy to provide the necessary efficacy.

### SUMMARY OF THE INVENTION

Accordingly, the present invention is a magnetic moxibustion T-shirt vest which includes: a T-shirt vest noumenon and multiple magnetic stripes fixed and distributed on the noumenon. The described T-shirt vest noumenon includes posterior film, left anterior film, right anterior film and sleeves. The described multiple magnetic stripes are set on the described T-shirt vest noumenon posterior film, left anterior film, right anterior film and sleeves based on the preset primary spacing, and the described magnetic stripes include substrate and multiple magnets set on the substrate based on the preset secondary spacing. The described multiple magnets are fixed on the substrate based on unipolar direction or bipolar direction. The magnetic moxibustion T-shirt vest has achieved magnetotherapy on the whole upper body, and has the function of mitigating and treating shoulder and cervical spondylosis. The present invention further achieves magnetotherapy on the entire upper body, and has the function of mitigating and treating shoulder and cervical spondylosis.

In the most preferred embodiment of the present invention, the T-shirt vest is worn like a standard T-shirt. The described multiple magnets are set on the substrate through the multiple grooves while every groove corresponds to one magnet, and there can be a protection layer which is set on the described substrate. The material of the substrate is soft and not magnetically permeable. Also, the described magnetic stripes can be set on the described T-shirt vest through sewing or adhesion. Also, there can be multiple grooves on the described T-shirt vest, and the magnetic stripes are then set in the grooves.

For the magnetic moxibustion T-shirt vest provided in the present invention, by setting multiple magnetic stripes on the T-shirt vest noumenon with multiple magnets on each stripe, the magnetic field produced by this structure can intensively act on a shoulder, waist and cervical vertebrae of the back, as well as breast, triceps, etc. in order to conduct magnetotherapy. The present invention can then achieve magnetotherapy for the whole upper body, and can mitigate and treat shoulder and cervical spondylosis. Moreover, the magnetic moxibustion T-shirt vest has a simple structure, is easy to use, and magnetotherpay can be conducted anytime and anywhere by wearing the T-shirt vest.

### BRIEF DESCRIPTION OF THE DRAWINGS

FIG. 1 is an illustration of the back view structure of the magnetic moxibustion T-shirt vest according to the present invention, which is worn in the same manner as wearing a standard T-shirt.

FIG. 2 is an illustration of the posterior film of the magnetic moxibustion T-shirt vest.

FIG. 3 is an illustration of the anterior film of the magnetic moxibustion T-shirt vest.

FIG. 4 is an illustration of the sleeves of the magnetic moxibustion T-shirt vest.

### DETAILED DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation or restriction of the scope of the invention is thereby intended. Any alterations and further implementations of the principles of the invention as described herein are contemplated as would normally occur to one skilled in the art to which the invention relates.

### First Preferred Embodiment

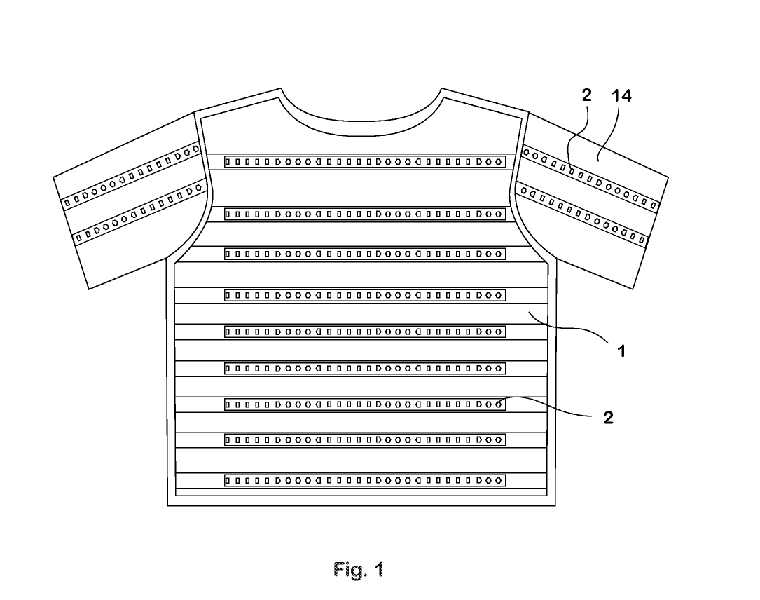

FIG. 1 is an illustration of the back view structure of the first most preferred embodiment of the magnetic moxibustion T-shirt. As shown in FIG. 1, the magnetic moxibustion T-shirt vest. The preset primary spacing can have different values, as long as the magnetic field strength produced by the stripes is relatively strong. The substrate is set on the T-shirt vest noumenon 1.

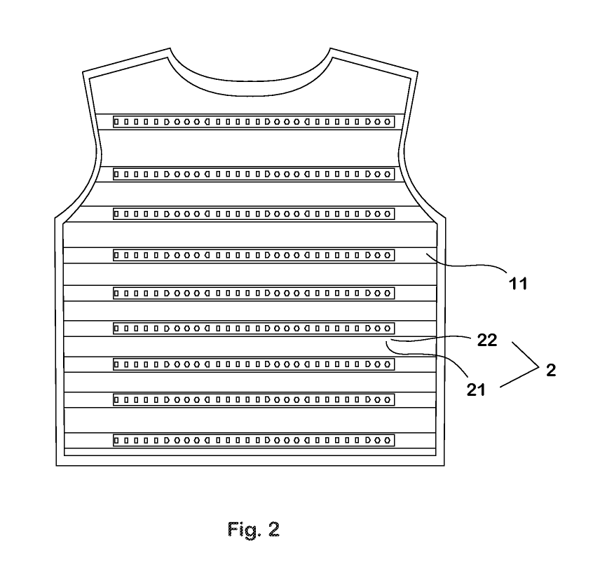

In this embodiment shown in FIG. 2, the T-shirt vest noumenon 1 can be knitted fabric, etc. For example, if multiple magnets 22 are set on the substrate 21 and the substrate 21 are then set on the posterior film 11, the left anterior film, the right anterior film, and sleeves 14 of the T-shirt vest noumenon 1, a type of magnetic moxibustion T-shirt best formed. In this embodiment, magnetic stripes 2 can be set on noumenon 1 through sewing or adhesion. A detailed fixation approach can be chosen based on practical application, and there is no restriction in this embodiment. In this embodiment, the multiple magnets 22 are magnetically permeable material. In order to prevent the disturbance of substrate on multiple magnetic stripes, material of the substrate is not magnetically permeable. It can also be a flexible substrate. The elongation direction of the above magnetic stripes can be along the horizontal direction. Specifically, multiple magnets can be arranged based on the unipolar direction when they are set on the substrate in this embodiment. They can also be arranged based on the bipolar direction. The unipolar direction arrangement means that the N pole or S pole of the multiple magnets has the same direction, while the bipolar direction arrangement means their N pole or S pole has different directions. In one example, the magnets can be arranged based on the bipolar direction and they can constitute a magnetic array group. Neighboring magnets in the described magnetic array group have mutually different polarity. Namely, the S pole is adjacent to the N pole, and the N pole is adjacent to the S pole.

Heat treatment can be conducted on the substrate in other embodiments as well in order to obtain a hot magnetic moxibustion T-shirt vest combined with high temperature and strong magnetic forces. This can conduct fomentation and magnetotherapy on users to achieve health care while the temperature for fomentation can be automatically controlled.

The magnetic field produced by multiple magnets 22 in this structure can act on the whole back (including shoulder, waist and cervical vertebrae), as well as chest and triceps, in order to conduct magnetotherapy on the whole upper body. This can activate cells, enhance immune functions, improve microcirculation, and relieve pain. Etc. Thus, should and cervical spondylosis can be mitigated and treated.

As the magnetic moxibustion T-shirt vest has sleeves, it can conduct magnetotherapy on arms. In this embodiment, during application of the magnetic moxibustion T-shirt vest, the multiple magnetic stripes are corresponding to the body's acupoints. Thus, the position of the magnetic stripes can be changed based on the actual application. Moreover, there is no side effect for magnetotherapy. In this embodiment, material of the substrate is soft, and the magnets can be set on the substrate through sewing or adhesion. The detailed approach should be chosen based on practical application and there are no restrictions on the use. The shape of the substrates can be rectangular as shown in this FIG. 1 as well as other irregular shapes.

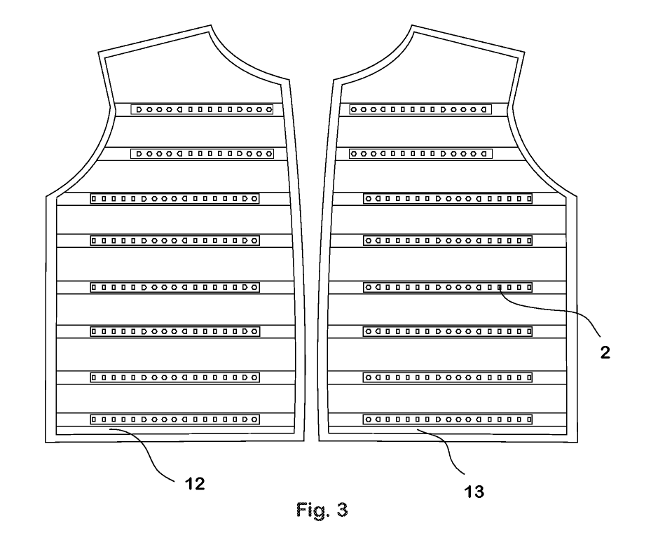

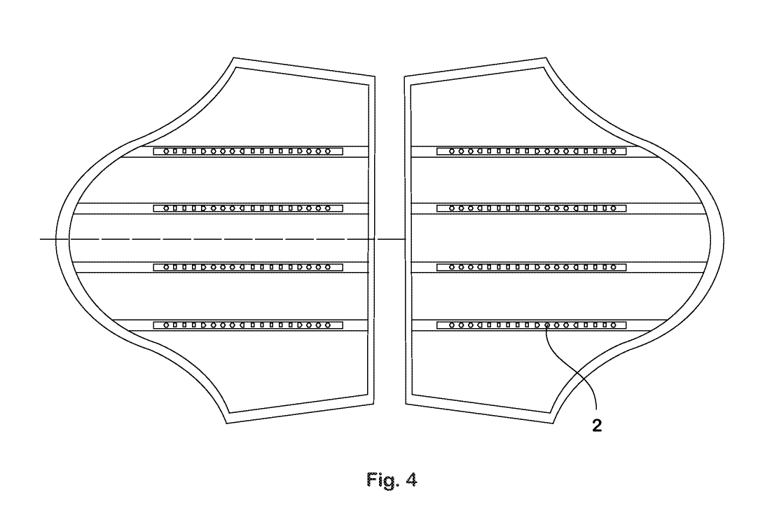

FIG. 3 shows the left anterior film 12 and right anterior film 13. Yet in FIG. 4, the sleeves are shown before being fixed on the T-shirt vest noumenon. The length of the magnetic stripes in this embodiment can be set based on detailed size of the T-shirt vest. For example, the length can be 140 mm, 150 mm, 160 mm, 170 mm, 190 mm, 330 mm, 375 mm, 390 mm, etc. The distance between magnetic stripes can also be changed based on actual situation, for example 65 mm. For the magnetic moxibustion T-shirt vest provided by the present invention, by setting multiple magnetic stripes on the T-shirt vest noumenon with multiple magnets on each stripe, the magnetic field produced by this structure can intensively act on a shoulder, waist, cervical vertebrae of the back, as well as breast, triceps. etch in order to conduct magnetotherapy. It has achieved magnetotherapy for the whole upper body, and can mitigate and treat shoulder and cervical spondylosis, etc. Moreover, the magnetic moxibustion T-shirt vest has simple structure and is easy to use, in which case magnetotherapy for the whole upper body can be conducted anytime and anywhere.

Furthermore, based on the above embodiments, there are multiple grooves on the T-shirt vest noumenon. The described magnetic stripes are set on the described grooves. Specifically, every good is corresponding to one magnetic stripe. Alternatively, multiple connectors can be set on the grooves, and multiple magnetic stripes can be fixed on the noumenon through these connectors. Magnetic stripes can also be set in the grooves through sewing or adhesion. Furthermore, based on above embodiments, multiple grooves are set on the substrate, and magnets are set in the grooves. Namely, grooves and magnets are in correspondence one to one. The multiple magnets can also be placed into one groove, and detailed approach should be chosen based on practical application. In this embodiment, the shape of magnets is round with a diameter of 5-10 mm, while they can also be other shapes as well.

Furthermore, based on above embodiments, multiple magnets are equally spaced on the substrate to produce more even magnetic field strength. In this case, magnetic field strength acting on some areas of the back is uniform, which can better satisfy users' needs. In addition, the distance between the magnets is 10-30 mm, but the distance can also be 20 mm or 30 mm. A protection layer, which is on the substrate, and the multiple magnets are between the protection layer and substrate. By setting a protection layer on the substrate, magnets are protected, preventing their shedding from substrate during application. Magnets are also better fixed and protected by being set between the substrate and the protection layer.

Although one or more embodiments of the newly improved invention have been described in detail, one of ordinary skill in the art will appreciate the modifications to the material selection and utility functions of the moxibustion T-shirt vest along with the new magnetic stripe layout of the magnetic moxibustion T-shirt vest. In particular, by using the layout of the multiple magnets distributed across sections of the vest designed to provide moxibustion therapy to the upper body parts of the user, a desirable assembly and more effective footprint is created to enhance magnetic moxibustion therapy for humans. It is acknowledged that obvious modifications will ensue to a person skilled in the art. The claims which follow will set out the full scope of the claims.

1. A magnetic moxibustion T-shirt vest comprising: a T-shirt vest noumenon, magnets, multiple magnetic stripes, a substrate, sleeves, posterior film, left anterior film, right anterior film, and multiple magnets inside the multiple magnetic stripes, posterior film, left anterior film, right anterior film, wherein the multiple magnetic stripes are fixed and distributed on the vest noumenon posterior film, left anterior film, right anterior film and collar, the multiple magnetic stripes are set and based on a preset primary spacing, and the magnetic stripes include the substrate and multiple magnets set on the substrate based on a preset secondary spacing, wherein the multiple magnets are fixed and distributed on the substrate based on a unipolar and bipolar direction.

2. The magnetic moxibustion T-shirt vest of claim 1 wherein the multiple magnets are set on the substrate through multiple grooves on the substrate and every groove corresponds to one magnet.

3. The magnetic moxibustion T-shirt vest of claim 1 wherein the multiple magnetic stripes can be fixed on the noumenon by sewing or adhesion.

4. The magnetic moxibustion T-shirt vest of claim 1 wherein the noumenon has multiple grooves and the multiple magnetic stripes are set into the grooves.

5. The magnetic moxibustion T-shirt vest of claim 1 wherein the substrate is composed of material that is soft and not magnetically permeable.

6. The magnetic T-shirt vest of claim 1 wherein the distance between the magnets on the substrate are set between 10-30 mm apart.

7. The magnetic T-shirt vest of claim 6 further comprising a protection layer which is set on the substrate and the multiple magnets are between the protection layer and substrate.
